(19) United States
(12) Patent Application Publication
(10) Pub. No.: US 2022/0218990 A1
(43) Pub. Date: Jul. 14, 2022

(54) METHOD AND SYSTEM FOR REGULATION AND RESTORATION OF INDEPENDENT STEPPING FOR PATIENTS WITH MOTOR PATHOLOGY

(71) Applicant: COSYMA LTD., Moscow (RU)

(72) Inventors: Yuri Petrovich GERASIMENKO, P/o Koltushi (RU); Tatiana Romulievna MOSHONKINA, Sankt-Peterburg (RU); Alexandr Alekseevich GRISHIN, Moscow (RU)

(21) Appl. No.: 17/607,499

(22) PCT Filed: Feb. 5, 2020
(86) PCT No.: PCT/RU2020/050012

§ 371 Date: Oct. 29, 2021

(30) Foreign Application Priority Data
May 20, 2019 (RU) 2019114792

(51) Int. Cl. A61N 1/36; A61N 1/04

(52) U.S. Cl. CPC A61N 1/36003; A61N 1/0456; A61N 1/36034; A61N 1/36014

(57) ABSTRACT

The present invention relates to restoration of motor activity for dysfunctions of gait. A method is provided using electrical stimulation of the spinal cord, including simultaneous continuous stimulation of the spinal cord at least at the level of T11-T12 vertebrae and spatio-temporal and spatio-selective stimulation of the spinal cord roots at the level of T11 and L1 vertebrae. A neuroprosthesis system is provided, including a multi-channel stimulator for transcutaneous electrical stimulation of the spinal cord that includes at least one storage device and one or more programs that are loaded into at least one storage device, with one or more programs containing instructions, an electrode or electrode array connected to the stimulator, at least one recording device to detect the contact and/or detachment of a lower limb and/or a walking aid on which the upper limb leans during locomotion, and a device for triggering or cessation of stimulation

### FIELD

The present disclosure relates to medicine and medical equipment, and more specifically to neurophysiology and rehabilitation.

### BACKGROUND

Invasive methods of stimulation of spinal cord of monkeys with hind limb paraplegia (Marco Capogrosso et al. A brain-spine interface alleviating gait deficits after spinal cord injury in primates. Nature, volume 539, pages 284-288 (2016)) and rats with hind limb paraplegia (Parag Gad et al. Forelimb EMG-based trigger to control an electronic spinal bridge to enable hindlimb stepping after a complete spinal cord lesion in rats. Journal of NeuroEngineering and Rehabilitation (2012)) are known. Rhythmic electrical stimulation of the motor pools of the flexor muscles and the extensor muscles of the hind limbs was triggered by signals of the cerebral cortex, which detected the phases of the locomotion cycle in monkeys with unilateral transection (hemisection) of the spinal cord.

The movements of the paralyzed hind limbs were initiated by electrical stimulation of the lumbar region of the spinal cord by signals from the muscles of the forelimbs in rats moving along the treadmill with a completely cut spinal cord in the thoracic region. The movements of the upper and hind limbs of the rats were found to be uncoordinated as a result of stimulation of the spinal cord. The problem of transformation of the command from the brain to the motor pool of hindlimbs bypassing the spinal cord injury separating these areas was solved in both cases. The locomotor function is regulated by stimulation of the spinal cord below the lesion level.

Both methods described above are invasive techniques of stimulation associated with surgeries on the brain and spinal cord (Marco Capogrosso et al.) or on the spinal cord and forelimb muscles of animals (Parag Gad et al.). An invasive method of stimulation is associated with the necessity of postoperative care, the risk of inflammatory reactions and the probability of rejection of implanted devices. Moreover, locomotion in both cases is realized only in the process of stimulation of the spinal cord, which indicates the prosthetics of locomotor function, and not the restoration of this function.

It is known that non-invasive magnetic stimulation of the spinal cord in a patient can be initiated by rhythmic arm movements which trigger rhythmic electromagnetic stimulation of the spinal cord (Syusaku Sasada et al. Volitional Walking via Upper Limb Muscle-Controlled Stimulation of the Lumbar Locomotor Center in Man. Journal of Neuroscience, 13 Aug. 2014, 34 (33)).

Magnetic stimulation has lower precision (targeting) than electrical stimulation. The magnetic coil, which generates a magnetic field to impact the spinal cord, has a significant mass and complex shape. Therefore, either an additional fixing device or an assistant holding the coil is required to fix it next to the spinal cord. Therefore, magnetic stimulation cannot provide an independent stepping of the patient on an even surface. Also magnetic stimulation of the spinal cord during even a short period of time of locomotor activity (the time of locomotor activity coincides with the duration of the magnetic stimulation) uses a higher signal strength, requiring a giant battery capacity compared to electrical stimulation.

More recently, electrical epidural stimulation of the spinal cord, which allows control of stepping performance in people who have suffered a spinal cord injury more than four years prior and have a permanent motor deficit or complete paralysis, despite intensive rehabilitation. (Targeted neurotechnology restores independent stepping in humans with spinal cord injury. Nature, volume 563, pages 65-71 (2018)). Spinal cord stimulation was performed using an implanted pulse generator with the ability to trigger real-time stimulation and an electrode array placed on the posterior surface of the spinal cord in the lumbar-sacral area. Information about the kinematics of patients' stepping was processed in real time, including measurements of electrical activity of the leg muscles (electromyography, EMG).

Spatio-temporal selective stimulation of the dorsal roots of the spinal cord, synchronized with a certain phase (stance, or limb transfer) of the stepping cycle, was performed to activate the motor pools of flexor muscles and extensor muscles at the intervals necessary for stepping performance. Stepping characteristics improved during the rehabilitation process. After a few months, patients with incomplete lesions of the spinal cord restored arbitrary control over previously paralyzed muscles without stimulation; patients were able to walk or ride a bicycle outside the laboratory during the performance of stimulation of the spinal cord.

The use of such stimulation is limited by the number of patients who are not contraindicated by neurosurgical operation for the implantation of epidural electrodes. Any surgical operation is associated with the risks of the surgery itself, the risks of postsurgery complications, the risk of rejection of implantable devices, the necessity of postsurgery care, which duration depends on a large number of factors. Moreover, the stimulation described above does not solve the problem of transmitting the command to start and control stepping from the brain to the leg motor pools, bypassing the spinal cord injury that separates these sites. This locomotion is a “mechanical” one, which is not adapted to the environmental conditions, due to the impossibility of controlling of locomotion by brain during such stimulation.

### SUMMARY

The purpose of the present invention is improvement of neurological restoration of motor activity and improvement of quality of life of patients with diseases and traumatic injuries of the brain and/or spinal cord, the consequence of which is a dysfunction of the gait as well as increasing the efficiency and reduction of the rehabilitation period, providing the possibility of independent stepping of patients outside the hospital conditions.

The technical result achieved by the invention is the development of a method of non-invasive (not requiring surgery) spatio-temporal electrical stimulation of the spinal cord:
which allows to use the natural synergies in the movements of the upper and lower limbs for the realization of stepping function in patients with disorders of this function;
in which the command to start the spinal stimulation, which initiates stepping , from the movements of the upper body parts (arms, head, torso) and control of the stepping performance is implemented;
which realizes the possibility of simultaneous stimulation of the spinal cord at different levels according to different selected algorithms, which allows the use of simultaneous stimulation of cervical enlargement to ensure an increase in the activity of the arms; in the area of lumbar enlargement—to increase its excitability.
which allows to facilitate voluntary stepping, and also to restore and provide independent stepping of patients with disorders caused by diseases and/or traumatic damage of the brain and/or spinal cord, and allows to restore motor function of the upper and lower limbs .

Another technical result achieved by the invention is the development of a device to implement the method described above.

The technical result of the invention is achieved through a method of facilitating voluntary independent stepping using transcutaneous electrical stimulation of the spinal cord including simultaneous continuous stimulation of the spinal cord at least at the level of the T₁₁-T12 vertebrae and spatio-temporal and spatio-selective stimulation of the roots of the spinal cord at the level of the T₁₁ and L1 vertebrae in patients with traumatic injuries and/or diseases of the spinal cord and/or brain.

Wherein, the triggering and cessation of transcutaneous electrical stimulation of the spinal cord is performed through external control.

In some embodiments, one of the movements performed to control the device for triggering or cessation of stimulation can be chosen as an external control: movement of at least one intact upper or lower limb, head movement, shoulder lift, torso movement.

Moreover, the trigger of spatio-temporal and spatio-selective stimulation of the roots of the spinal cord at the level of the T11 and L1 vertebrae is the motion of the intact upper or lower limb.

Current intensity in transcutaneous electrical stimulation is selected individually depending on the excitability and threshold of the motor response and pain sensitivity of the patient. In some embodiments, monopolar rectangular pulses or bipolar rectangular pulses with a carrier frequency in the range from 5 kHz to 10 kHz are used for transcutaneous electrical stimulation of the spinal cord, with a stimulation frequency for continuous stimulation of the spinal cord at least at the level of the T11-T12 vertebrae chosen in the range from 30 Hz to 45 Hz, the stimulation frequency for stimulation of the roots of the spinal cord at the level of the L1 vertebrae is chosen in the range of 10 Hz to 30 Hz, the stimulation frequency for stimulation of the roots of the spinal cord at the level of the T11 vertebrae is chosen in the range of 30 Hz to 50 Hz. The indifferent electrodes (anodes) are located on the skin above the crests of the iliac bones or on the abdomen. The anodes are symmetrically located in the left and right clavicle region or on the crests of the iliac bones during stimulation of the cervical level of the spinal cord.

In further embodiments, the stimulations are performed in patients with hemiparesis, wherein, the patient walks over ground or moving treadmill with the upper limb leaning on a fixed support (handrails) or with partial compensation of body weight by means of a suspension system during continuous stimulation of the spinal cord and spatio-temporal and spatio-selective stimulation of the roots of the spinal cord.

In further embodiments, the method includes the following steps:

1) triggering and activation of continuous stimulation at least at the level of the T11-T12 vertebrae and triggering of spatio-temporal and spatio-selective stimulation of the roots of the spinal cord at the level of the T11 and L1 vertebrae by means of external control, which initiates the start of stimulation;

2) activation of stimulation of the roots of the spinal cord on the lesion side at the level of the L1 vertebrae at the moment when the intact lower limb starts the swing phase.

3) cessation of the stimulation of the roots of the spinal cord on the lesion side at the level of the L1 vertebrae with simultaneous activation of the stimulation of the roots of the spinal cord on the lesion side at the level of the T11 vertebrae at the moment when intact lower limb starts the stance phase.

4) stimulation of the roots of the spinal cord on the lesion side at the level of the T11 vertebrae when the paretic lower limb starts the swing phase.

5) cessation of the stimulation of the roots of the spinal cord on the lesion side at the level of the T11 vertebrae when the paretic lower limb starts the stance phase.

6) repeating steps 2)-5) any amount of times;

7) cessation of continuous stimulation of the spinal cord at least at the level of the T11-T12 vertebrae and cessation of spatio-temporal and spatio-selective stimulation of the roots of the spinal cord at the level of the T11 and L1 vertebrae due to external control, which stops stimulation.

Moreover, continuous stimulation of the spinal cord may be additionally performed at the level of the C5-C6 vertebrae in the implementation of the steps of the method described above.

In further embodiments, a patient with hemiparesis performs over ground stepping or stepping on a moving treadmill during continuous stimulation of the spinal cord and spatio-temporal and spatio-selective stimulations of the roots of the spinal cord using a support device located in an intact upper limb.

In some embodiments, a walking aid is selected from: a walking stick, a cane, a crutch.

In some embodiments, the patient performs stepping movements using a suspension system.

In further embodiments, the method includes the following steps:

1) triggering and activation of continuous stimulation at least at the level of the T11-T12 vertebrae and start of spatio-temporal and spatio-selective stimulation of the roots of the spinal cord at the level of the T11 and L1 vertebrae by means of external control, which initiates the beginning of stimulation;

2) activation of stimulation of the roots of the spinal cord on the lesion side at the level of the L1 vertebrae at the moment when the intact lower limb starts the swing phase.

3) cessation of the stimulation of the roots of the spinal cord on the lesion side at the level of the L1 vertebrae at the moment when intact lower limb starts the stance phase.

4) activation of stimulation of the roots of the spinal cord on the lesion side at the level of the T11 vertebrae at the moment of lifting of walking aid from the surface.

5) stimulation of the roots of the spinal cord on the lesion side at the level of the T11 vertebrae when the paretic lower limb starts the swing phase.

6) deactivation of stimulation of the roots of the spinal cord on the lesion side at the level of the T11 vertebrae at the moment when the paretic lower limb start stance phase.

7) repeating steps 2)-6) any amount of times;

8) cessation of continuous stimulation of the spinal cord at the level of the T11-T12 vertebrae and at the level of the C5-C6 vertebrae and cessation of spatio-temporal and spatio-selective stimulation of the roots of the spinal cord at the level of the T11 and L1 vertebrae occurs due to external control, which stops the stimulation.

Moreover, continuous stimulation of the spinal cord can be performed additionally at the level of the C5-C6 vertebrae. The triggering and activation of continuous stimulation is first performed at the level of the T11-T12 vertebrae, and then at the level of the C5-C6 vertebrae. The transcutaneous electrical stimulation is performed using a multi-channel stimulator.

In some embodiments, at least one recording device is used to detect:
the contact of the lower limb or the walking aid on which the intact upper limb leans, with the surface;
the detachment of the lower limb or the walking aid on which the intact upper limb leans, from the surface;
   and at least one recording device transmits the control signal to the stimulator when the above events are detected.

In some embodiments, the stimulator contains at least one storage device and one or more programs that are loaded into at least one of the above-mentioned storage devices, with one or more programs containing instructions for: triggering and cessation of stimulation in dependence of the control signal received from the device for triggering or cessation of stimulation during external control; activation and deactivation of continuous stimulation by regulating the supply of electrical current to the corresponding electrodes, in dependence of the control signal received from the device for triggering or cessation of stimulation during external control; activation and deactivation of the spatio-temporal and spatio-selective stimulations of the roots of the spinal cord by regulating the supply of electrical current to the corresponding electrodes, in dependence of the control signal received from at least one device for recording the detection of contact and/or detachment of the intact and/or paretic lower limb and/or the walking aid on which the intact upper limb leans.

The technical result is also achieved in that the spinal neuroprosthesis, which facilitates voluntary stepping performance in patients with traumatic injuries and/or diseases of the spinal cord or brain, contains a multi-channel stimulator for transcutaneous electrical stimulation of the spinal cord that includes at least one storage device and one or more programs that are loaded into at least one storage device, with one or more programs containing instructions for the implementation of the method described above, and electrodes connected to the specified stimulator, at least one recording device to detect the contact and/or detachment of the intact and/or paretic lower limb and/or the walking aid on which the intact upper limb leans during stepping, a device for triggering or cessation of stimulation.

In addition, at least one recording device may be selected from: surface contact sensor, surface contact sensor of the walking aid.

In addition, the device for triggering or cessation of stimulation may be made in the form of an electromechanical switch or radio-frequency switch.

In addition, the spinal neuroprosthesis may include an acceleration sensor and/or angular velocity sensor and/or a joint angle sensor.

### BRIEF DESCRIPTION OF THE DRAWINGS

The features of the invention will become more evident on the basis of the following detailed description, in which reference is made to the attached figures, as follows:

FIG. 1 shows a position of a subject in a biomechanical training machine for neurorehabilitation of motor and visceral functions.

FIG. 2 depicts a scheme for conducting a study of how voluntary arm movements affect the characteristics of involuntary leg movements caused by transcutaneous electrical stimulation of spinal cord (TESSC).

FIG. 3 shows recordings of how arm movements affect the characteristics of involuntary leg movements caused by TESSC.

FIG. 4 shows recordings and results of an analysis of the effect of arm movements on the characteristics of movements caused by TESSC.

FIG. 5 shows a position of a subject during a study of how periodic TESSC applied to different roots of the spinal cord in different phases of the step (spatio-temporal TESSC) modulates characteristics of human step movements.

FIG. 6 shows cathode location in a projection of the spinal cord and the roots of the spinal cord during studies of the impact of spatio-temporal TESSC on the characteristics of human step movements.

FIG. 7 illustrates EMG activity of the muscles of the legs and angles in the knee joint during treadmill stepping of a subject

FIG. 8 shows movement trajectories of the legs during treadmill stepping without stimulation and with continuous and periodic TESSC at the different levels.

### DETAILED DESCRIPTION

### Terms and Definitions

Definitions of some of the terms used in this description are listed below. Technical and scientific terms in this application have standard meanings, generally accepted in the scientific and technical literature, unless specified otherwise.

The terms “includes”, “including” and “included”, “having”, “supplied”, “containing” and their other grammatical forms are not intended to be interpreted in an exclusive sense, but, on the contrary, are used in a non-exclusive sense (i.e. in the sense of “having in its composition”).

The terms “regulation of locomotion”, “control of locomotion” means control of stepping characteristics in general (speed, step amplitude, duration), and its components, for example, phases of a step cycle (swing, stance).

“Facilitation of locomotion” means a particular case of locomotion regulation where managing locomotion characteristics leads to improving coordination, gait stability and locomotion speed.

The term “stimulation” relates to the electrical impacts of alternating current, wherein “continuous stimulation” means stimulation, the beginning and the end of which do not depend on the rhythmic movements of intact (not affected by the pathological process that caused the hemiplegia) arms and legs; “intermittent (rhythmic) stimulation” means a stimulation, the beginning and the end of which are synchronized with the rhythmic movements of intact arms and legs, wherein “spatio-selective stimulation” is understood as intermittent (rhythmic) stimulation, at which the level of stimulation and lateralization (right or left) of stimulation depends on the phase of movements of intact arms and legs, “spatio-temporal stimulation” means spatio-selective stimulation, in which the beginning and end of this stimulation depends on the phase of movements of intact arms and legs.

The terms “synergies”, “motor synergies”, “synergies of movements” refers to associated and coordinated contraction and relaxation of the muscles of the arms and legs during the step movements. It is shown that the control of human and animal movements is realized not as the control of individual muscles, but as the control of synergies of movements (Aleksandrov et al. Sustainable control of the posture and movements of a standing humanoid based on the principle of natural human synergies. Russian Journal of Biomechanics. 2013. B. 17, No 1 (59): 94-109).

The term “activation of motor synergies” refers to the associated and coordinated contraction and relaxation of the groups of the legs muscles when performing movements of the arms and other moving parts of the body. An advantage of activation of synergies is facilitation of movements caused by transcutaneous stimulation of the spinal cord (TESSC), activation of intrinsic mechanisms for controlling movements.

Examples of diseases of the brain and/or spinal cord are brain or spinal stroke, degenerative and inflammatory diseases of the brain or spinal cord, iatrogenic diseases, resulting in impaired locomotor function. Examples of damage to the brain and/or spinal cord are concussion of the brain, compression of the brain, brain injury, brain hemorrhage due to impact on the head, concussion of the spinal cord, injury of the spinal cord, compression of the spinal cord, anatomical rupture of the spinal cord, spinal cord hematomyelia, spinal cord hematoraxis, damage to the main vessel of the spinal cord, damage to the roots of the spinal nerves.

In addition, the terms “first”, “second”, “third”, etc. are used simply as conditional markers, without imposing any numerical, sequential or other restrictions on the listed objects.

The term “connected” means functionally interconnected, and any number or combination of intermediate elements between the interconnected components (including the absence of intermediate elements) may be used.

Human locomotion involves the coordinated movements of all four limbs, wherein neural networks controlling the movements of upper limbs closely interact with neural networks controlling locomotor synergies of lower limbs (Zehr E. P., Duysens J. Regulation of arm and leg movement during human locomotion//Neuroscientist. 2004. V. 10. No. 4P. 347; Selionov V. A. et al. INTERLIMB INTERACTIONS DURING CYCLIC IN-PHASE AND ANTIPHASE MOVEMENTS OF ARMS AND LEGS AND THEIR DEPENDENCE ON AFFERENT INFLUENCES//Human Physiology.—2014. V. 40.-NO. 4.-P. 410-421.)

Arm movements have a modulating effect on leg movements while walking, despite the fact that humans are characterized by bipedal locomotion, unlike most other mammals. There were significant changes in the parameters of leg muscle reflexes recorded in the conditions when the subjects walked on the treadmill were asked to make movements in the rhythm of locomotion. The values of the measured characteristics of these reflexes were influenced by the phase and velocity of arm movements, as well as other characteristics of arm movements. (Zehr EP, Chua R. 2000. Modulation of human cutaneous reflexes during rhythmic cyclical arm movement. Exp Brain Res 135:241-50; Zehr E P, Kido A. 2001. Neural control of rhythmic, cyclical human arm movement: task dependency, nerve specificity and phase modulation of cutaneous reflexes. J Physiol (Lond) 537:1033-45; Zehr E P, Collins D F, Frigon A, Hoogenboom N. 2003. Neural control of rhythmic human arm movement: phase dependence and task modulation of Hoffmann reflexes in forearm muscles. J Neurophysiol 89:12-21.).

Studies on healthy volunteers showed that arm movements during treadmill stepping at speeds of 1-7 km/h increased stability if the right and left arms moved intensively in anti-phase with the same legs, but did not affect stability if they did not move while walking or moved in-phase with the same legs (Punt M. et al. Effect of arm swing strategy on local dynamic stability of human gait//Gait & posture. -2015.-V. 41.-No. 2.-P. 504-509).

Research has also shown that the spatial organization of human limb movements is an essential factor determining muscle activity. (Selionov V. A. et al. INTERLIMB INTERACTIONS DURING CYCLIC IN-PHASE AND ANTIPHASE MOVEMENTS OF ARMS AND LEGS AND THEIR DEPENDENCE ON AFFERENT INFLUENCES//Human Physiology.—2014.-V. 40-No. 4.-P. 410-421).

The activity of the muscles of the upper and lower limbs during separate and combined cyclic movements of arms and legs with different phase relations between the movements of the limbs were recorded in 10 healthy subjects in a prone position. Anti-phase active arm movements were characterized by greater muscle activity than in-phase. A significant increase in activity was observed in the biceps muscle of arm, tibalis anterior muscle and biceps muscle of thigh during a motor task that implements combined anti-phase movements of both upper and lower limbs, as compared to a motor task that implements their combined in-phase movements.

It has been shown that arm training in the process of motor rehabilitation of stroke patients affects the quality of stepping performance due to the activation of interneuronal connections (Kaupp C, Pearcey G E, Klarner T, Sun Y, Cullen H, Barss T S, Zehr E P. Rhythmic arm cycling training improves stepping performance and neurophysiological integrity in chronic stroke: the arms can give legs a helping hand in rehabilitation. J Neurophysiol 119: 1095-1112, 2018). Cyclic arm movements of these patients modulated the size of the reflex motor responses of the leg muscles, increased the strength of the leg muscles, coordination of muscle activity during stepping, and coordination between the arms and legs during locomotion.

The present invention is aimed at a system and method of regulation and restoration of independent stepping in patients with motor pathology of various genesis by using multisegmentary electrical transcutaneous spinal cord stimulation (TESSC) and activation of intrinsic interlimb motor synergies.

The transcutaneous electrical stimulation method allows activation of the spinal cord in several segments simultaneously. Therefore, there is a possibility of simultaneous direct and indirect activation of spinal structures regulating locomotion. Direct rhythmic stimulation is associated with the targeted activation of the motor pools of the muscle groups involved in the different phases of the step. Continuous stimulation is addressed to the neural locomotor network, which generates a locomotor pattern, i.e. sets the rhythm and determines the structure of motion. Thus, there is a combined and coordinated contraction and relaxation of muscle groups of affected lower and/or upper limbs during the movements of non-affected upper and/or lower limbs (arms or legs) facilitated by stimulation, which includes intrinsic movement control mechanisms.

Stimulation consists of continuous stimulation and intermittent (rhythmic) stimulation.

Continuous stimulation is provided to facilitate the movement of intact limbs. Continuous stimulation of the lumbar enlargement of the spinal cord is used at the level of the T11-T12 vertebrae to facilitate leg movements. Continuous stimulation of the cervical region of the spinal cord (cervical enlargement) at the level of the C5-C6 vertebrae is performed additionally if necessary to increase arm activity. Additional continuous stimulation may also be used in the thoracic and/or sacral regions of the spinal cord.

Intermittent (rhythmic) stimulation of the roots of the spinal cord at the level of the T11 and L1 vertebrae occurs in the presence of continuous stimulation of at least one of the above mentioned spinal cord regions to implement a particular type or phase of motion that is synchronized with the rhythmic movements of intact upper and/or lower limbs (arms and legs).

Rhythmic or continuous stimulation modes are prescribed by a specialist/physician and are determined by the pathology and characteristics of the movements of intact parts of the body.

The mode of stimulation in general, as well as the step, consists of several phases characterizing the process of stepping performance. The step consists of the propulsion phase, the swing phase and the stance phase (according to the kinematics of the leg movements). Information about the specified phases of the step is detected, wherein the spatio-selective rhythmic stimulation of the locus of the spinal cord is synchronized with the type of movements that are performed in a given period of time. The phase of movements of intact arms and legs (spatio-temporal rhythmic stimulation) determines the beginning and end of this stimulation.

Information about the step phases is required to activate the stimulation of the spinal cord at the right place at the right time: extensor muscles need to be activated during the stance phase, flexor muscles need to be activated during the swing phase. So, the rhythmic stimulation of the roots of the upper segments of the lumbar enlargement of the spinal cord is performed to activate the flexor muscles during the detectable propulsion and swing phases. In the detectable stance phase, the roots of the lower segments of the lumbar enlargement of the spinal cord are stimulated to activate the extensor muscles of the legs. The roots of the spinal cord on the right side are stimulated to regulate the movements of the right leg, and the roots of the spinal cord on the left side are stimulated to regulate the movements of the left leg. The accuracy of transcutaneous “to the root” stimulation makes it possible to cause the activity of muscles to be targeted: the activity of the muscles on the right or left, the activity of the muscles of the flexors and extensors.

The duration of individual phases of rhythmic stimulation is determined by the characteristics of the gait and movements of the intact upper and/or lower limbs of the patient, in particular, depend on the phase and velocity of movement of the upper and/or lower limbs of the patient.

Transcutaneous electrical stimulation of the spinal cord is implemented using monopolar rectangular pulses or bipolar rectangular pulses with an amplitude of current from 1 to 200 mA and a carrier frequency in the range from 5 kHz to 10 kHz.

In preferred embodiments, the amplitude of the current does not exceed 100 mA, as it is intended to use stimulation of the spinal cord in patients with intact sensitivity.

The intensity of the current during stimulation is prescribed by a specialist/physician, but may be corrected by the patient within a limited range. The intensity of the current is selected individually depending on the excitability, the individual value of the threshold of the motor response and the pain sensitivity of the patient.

The frequency of transcutaneous electrical stimulation lies in the range of 1-99 Hz. In some cases, the frequency of continuous stimulation of the spinal cord at least at the level of the T11-T12 vertebrae is chosen in the range from 30 Hz to 45 Hz. In some cases, the frequency of stimulation is chosen in the range from 30 Hz to 45 Hz if simultaneous continuous stimulation of the spinal cord at the level of the C5-C6 and T11-T12 vertebrae is required. In particular cases, the frequency of rhythmic stimulation of the roots of the spinal cord at the level of the vertebra L1 is chosen in the range from 10 Hz to 30 Hz. In particular cases, the frequency of rhythmic stimulation of the roots of the spinal cord at the level of the T11 vertebra is chosen in the range from 30 Hz to 50 Hz.

Multi-segmental stimulation exposure on the structures of the spinal cord is performed through separate electrodes or an electrode array fixed in the region of the patient's spine. The electrode array for transcutaneous stimulation of the spinal cord can be placed on the skin over the spine at the thoracic, cervical and/or lumbar region of the spinal cord in the projection of the corresponding neurons and neural networks of the spinal cord.

The triggering and cessation of stimulation may be carried out from a device for triggering or cessation of stimulation, which receives signals that characterize any external control. Movement of at least one of the intact upper/lower limbs, head movements, shoulder lift, torso movements and any other movement may be selected as external control.

For example, a movement in the shoulder joint, which is detected when leaning on a walking aid (where a walking aid comprises a stick, cane, crutch, etc.), which is held by the appropriate arm of the patient can act as an external control. This movement in the shoulder joint of the patient triggers stimulation, causing detachment of the contralateral leg from the surface.

For example, the movement of an intact upper limb (arm) can act as an external control in cases where at least one arm can move while stepping. In this case, the movement of at least one arm triggers stimulation and causes detachment of the contralateral leg from the surface.

In some cases, when the pathology of the injury is such that the patient can stand and walk a little bit, but the arms are not moving or amputated, it is possible to trigger the spatio-temporal stimulation of the spinal cord in any way available to the patient, for example by turning the head or lifting the shoulder, despite the fact that it is not possible to use the interlimb synergy.

The present invention is also directed to a spinal neuroprosthesis, with the help of which transcutaneous electrical stimulation of the spinal cord is performed and which regulates and restores independent stepping in patients with motor deficit of various genesis. Spinal neuroprosthesis is a means of transportation and rehabilitation, and its use not only leads to the restoration of normal stepping, but also to the restoration of mobility of the paretic arm in patients who have suffered a stroke.

Spinal neuroprosthesis is a complex of multichannel stimulators for transcutaneous electrical stimulation of the spinal cord and electrodes connected to the stimulator or electrode array attached to the skin over the patient's spine. Electrodes or electrode array are designed for repeated use and have a high conductivity. Also, the spinal prosthesis includes at least one recording device to detect a specific phase characterizing the stepping performance, and a device for triggering or cessation of stimulation.

The recording device is intended to detect contact with the surface of the lower limbs or the walking aid on which the intact upper limb leans, and detachment from the surface of the lower limbs or walking aid on which the intact upper limb leans, wherein the recording device is designed to transmit a control signal to the stimulator when the above events are detected.

The data transmission facilities are selected from the devices designed to implement the process of communication between different devices via wired and/or wireless communication. In particular, such devices can be: GPS modem, BLE module or Bluetooth, Wi-Fi transceiver, etc.

The stimulator provides the supply of electrical current of a certain form to the corresponding electrodes according to specified algorithms on the basis of the control signals received from the recording devices. Programs with the necessary instructions for performing the method of regulating and restoring independent stepping depending on the pathology of the motor activity of the patients are contained in at least one storage medium of the stimulator.

The storage device may be a hard disk drive (HDD), solid state drive (SSD), flash memory (NAND-flash, EEPROM, DataFlash, etc.), mini-drive or their combination.

The microcontroller performs the main computational work for the implementation of the algorithm for supplying electrical current to the electrodes depending on the received data. Components of the stimulator are interconnected via the data bus.

The microcontroller of the stimulator provides triggering and cessation of stimulation depending on the control signal received from the device for triggering or cessation of stimulation during the external control. In particular, the microcontroller provides the supply of electrical current of a given shape and frequency to one or another channel of the stimulator, and, consequently, to the corresponding electrode connected to it.

The microcontroller of the stimulator provides activation and cessation of continuous stimulation by regulating the supply of electric current to the corresponding electrodes depending on the received control signal from the device for triggering or cessation of stimulation during the external control.

The device for triggering or cessation of stimulation can be implemented using a button for pressing with the chin or shoulder, a walking aid, a myogram of the moving part of the patient's body and in any other way. The device for triggering or cessation of stimulation can be made in the form of an electromechanical switch or a radio-frequency switch.

The microcontroller of the stimulator provides switching on and off intermittent (rhythmic) stimulation of the roots of the spinal cord by regulating the supply of electric current to the corresponding electrodes depending on the control signal received from the device for detecting contact and/or detachment of the intact and/or parethic lower limb and/or the walking aid on which the intact upper limb leans.

The recording device can be made in the form of a sensor of contact with the surface or a sensor of contact of the walking aid with the surface.

The acceleration sensor and/or the angular velocity sensor and/or the angle change sensor in the joint, which are connected to the microcontroller by the data transmission device, can also be included in the spinal neuroprosthesis kit.

Further details of the steps of the method of regulation and restoration of independent stepping in patients is provided by an example of hemiparesis. Hemiparesis can occur as a separate phenomenon, or it is a stage of development of hemiplegia or it is detected when limb function is restored after hemiplegia.

The patient can walk on a fixed or moving treadmill with the intact upper limb leaning on a stationary support (handrails) during training. For example, the patient may be on a treadmill or in parallel bars when the healthy arm holds the bars on the training machine or on the floor when the healthy arm leans on the handrail.

In an alternative embodiment, when the patient is poorly coordinated, he can train independent stepping with partial compensation of body weight using a suspension system. Also, this type of training can be used by patients with upper paraparesis when both arms are paretic. For example, a patient can be secured with straps in the pelvic region and suspended in a vertical position so that the lower limbs are free, but at the same time leaned on the support. The patient's body is in the state of a pendulum.

In another alternative embodiment, the patient can walk on a fixed or on a movable surface with a walking aid, on which the relatively intact arm leans. Walking poles for Nordic walking, forearm crutches, orthopedic canes, etc. can be used as a means of walking aid.

The preparatory stage is carried out before the start of the training. The electrode array is placed on the skin, over the spine at the level of certain parts of the spinal cord, the electrodes within the array can be used independently, or separate electrodes can be placed for transcutaneous spinal cord stimulation (TSSC).

In more detail, the cathodes are placed and fixed in the middle line between the T11-T12 vertebrae and laterally, 1-3 cm back from the middle line, over the roots of the spinal cord from the affected side at the level of the T11 and L1 vertebrae. Common anodes for all these cathodes are located above the crests of the iliac bones only on the side of the cathode or on both sides.

Additionally, cathodes are placed and fixed in the middle line between the C5-C6 vertebrae, and anodes are placed over the crests of the iliac bones to stimulate the cervical enlargement. In the particular case (for more selective stimulation of the cervical enlargement) the anodes are located above the clavicle.

After installing the electrodes or the array of electrodes, they are connected to a multichannel stimulator for transcutaneous electrical stimulation.

The selection of the intensity of stimulation is carried out by a specialist/doctor and patient. The current intensity at continuous and rhythmic stimulation (30-40 Hz) should be at the level of parasthesia (tingling sensation, burning sensation, slight soreness, heaviness in the cathode region) or 5-10% less than this intensity, and should not cause discomfort or pain for stimulation using central cathodes placed in the middle line between the C5-C6 vertebrae and between the T11-T12 vertebrae.

Intensity of the current, causing contractions of the leg muscles for a single stimulation with a burst of pulses (1 ms) of 1 sec duration with a frequency of 10-50 Hz is used for stimulation using lateral cathodes (T11 and L1). Subsequently, currents of such intensity are used for walking, but this intensity should also not cause discomfort. Otherwise, the intensity should be reduced.

Selection of current occurs once, not before each use of spinal neuroprosthesis. Selection of current is repeated if the electrodes (cathodes/anodes) are changed or if the neuroprosthesis has not been used for a long time (from a week or more). Values of the selected electrical current intensities are stored in the data storage device of a multichannel stimulator for TESSC, included in the spinal neuroprosthesis. Starting stimulation is carried out in a standing or sitting position. Triggering is arbitrary due to the external control initiating the stimulation using the devise for triggering and cessation of stimulation described above. Stimulator channels for TESSC, supplying electrical current to the cathodes located at the level of the T11-T12, C5-C6 vertebrae are switched sequentially. Stimulation of these cathodes begins and terminates only after an arbitrary switching on and off, respectively. Further, the stimulator channels supplying electrical current to the lateral cathodes located over the roots of the spinal cord from the affected side at the level of the T11 and L1 vertebrae are triggered sequentially.

The intensity of electrical current on lateral cathodes at the initial moment of time is equal to zero. The stimulator channels connected to the lateral electrodes are switched on during movements of intact upper and/or lower parts of the body (conditionally healthy arms and legs), and modulated monopolar or bipolar pulses of electrical current of rectangular shape are applied to electrodes located above the roots of the spinal cord on the affected side on the level of the T11and L1 vertebrae. Thus, the arbitrary triggering of stimulation by the lateral cathodes is in fact the permission for stimulation by these cathodes.

At the same time, continuous stimulation at the level of the T11-T12, C5-C6 vertebrae is carried out throughout the entire training session. In one embodiment, the triggering and activation of continuous stimulation is performed only at the level of the T11-T12 vertebrae to activate neural locomotor networks of the spinal cord at the level of lumbar enlargement. In one embodiment, the triggering and activation of continuous stimulation is performed at the level of the T11-T12 and C5-C6 vertebrae for simultaneous activation of neural locomotor networks regulating the movement of the legs and arms, respectively.

In one embodiment, in the case when the patient is, for example, on a fixed surface leaning on an intact upper limb, the method of regulation and restoration of independent locomotion in a patient is as follows:

After the preparatory stage and the arbitrary triggering of stimulation, continuous stimulation is switched on at least at the level of the T11-T12 vertebrae and spatio-temporal and spatio-selective stimulation of spinal cord roots at the level of the T11 and L1 vertebrae is triggered.

The patient leans on a fixed surface with the intact upper limb and stands on a fixed surface. The initiation of walking is carried out from the standing position.

The patient makes the movement of the intact lower limb the same as when locomotion without letting go of the fixed walking aid, and transfers it forward, thereby transferring the weight of the body to the intact side. Stimulation of the roots of the spinal cord on the side of the lesion at the level of the L1 vertebrae is activated at the time when the intact lower limb starts swing phase. The duration of this stimulation is determined by the time from the moment of lifting of the intact lower limb from the surface until it is placed on the surface. As a result, the intact lower limb (a conditionally healthy leg) is placed in front of the paretic lower limb.

Stimulation along the lateral cathode T11 located over the roots of the spinal cord on the lesion side at the level of the T11 vertebrae is activated and lasts simultaneously with the termination of the stimulation of the roots of the spinal cord on the lesion side at the level of the L1 vertebrae when the intact lower limb has been placed on the surface. This stimulation causes the movement of the contralateral paretic lower limb, which is detached from the surface and transferred forward to the intact lower limb position, carrying the body weight. Stimulation of the roots of the spinal cord on the side of the lesion at the level of the T11 vertebrae is terminated when the paretic lower limb touches the surface.

This cycle may repeated any amount of times. The cessation of stimulation is carried out arbitrarily through external control by the device for triggering and cessation of stimulation. The patient sits down and disconnects the spinal neuroprosthesis at the termination of walking.

In one embodiment, in the case when the patient is, for example, on a fixed surface with a walking aid on which the intact upper limb of the patient leans, the method of regulation and restoration of independent stepping of the patient is carried out as follows:

After the preparatory stage and the arbitrary start of stimulation, continuous stimulation is activated at the level of the T11-T1 vertebrae and then, if necessary, at the level of the C5-C6 vertebrae, and spatio-temporal and spatio-selective stimulation of the roots of the spinal cord is triggered at the level of the T11 and L1 vertebrae. The patient leans on a walking aid with the intact upper limb and stands on a fixed surface. The intact upper limb on the conditionally healthy side of the patient's body leans on the walking aid (stick, cane, crutch). The walking aid is set on a fixed surface and is transferred slightly forward relative to the lower limbs of the patient for stability. The initiation of stepping performance is carried out from the standing position.

The patient makes a movement of the intact lower limb the same as when walking without letting go of the walking aid, and transfers it forward, thereby transferring the weight of the body to the intact side of his body. Stimulation of the roots of the spinal cord on the side of the lesion at the level of the L1 vertebrae is activated at the time of the detachment of the intact lower limb from the surface. The duration of this stimulation is determined by the time from the moment of lifting of the intact lower limb from the surface until it is placed on the surface. The stimulation of the roots of the spinal cord on the lesion side at the level of the L1 vertebrae is ceased after the intact lower limb has been placed on the surface. As a result, the intact lower limb (a conditionally healthy leg) is placed in front of the paretic lower limb and the walking aid.

Further, the intact upper limb, which leans on the walking aid, removes the walking aid from the surface and moves it forward slightly with respect to the intact lower limb. Stimulation by the lateral cathode T11, located above the roots of the spinal cord on the lesion side at the level of the T11 vertebrae, is activated at the moment of the detachment of the mean of walking aid from the surface. This stimulation causes the movement of the contralateral paretic lower limb, which is detached from the surface and carried forward to the position of the intact lower limb, carrying the body weight. Stimulation of the roots of the spinal cord on the side of the lesion at the level of the T11 vertebrae is switched off when the paretic lower limb touches the surface.

This cycle may be repeated any amount of times. The cessation of stimulation is carried out arbitrarily through external control by the device for triggering and cessation of stimulation.

The spinal neuroprosthesis is disconnected at the termination of walking.

The method described above can also be extended to paresis of the lower limbs while maintaining the function of supporting body weight, including with a walking aid (spinal cord injury with severity D, E on the ASIA scale, cerebral palsy with severity 1-3 on the GMFCS scale, brain injuries, demyelinating diseases, etc. with similar motor disorders).

Stimulation in this case will also be spatio-temporal. The triggering of stimulation can occur from intact upper limbs (arms). For example, the right arm triggers the stimulation of the muscles providing the stance phase and the transfer phase of the left leg, and symmetrically the left arm triggers the stimulation of the muscles providing the stance phase and the transfer phase of the right leg. It will also be possible to use this method with tetraparesis in cases where the patient has the opportunity to stand with or without a walking aid. The activity of the arms is increased by the use of the stimulation of the cervical region.

It is important to note that multisegmental non-invasive stimulation of the cervical and lumbar region of the spinal cord is performed for the regulation of interlimb synergies. Therefore, the stimulation of the cervical region will also restore the motor functions of the paretic arm, along with ensuring interlimb coordination. It has been shown that TESSC of the cervical region leads to the restoration of the functions of the upper limbs in paralyzed patients (Inanici F. et al. Transcutaneous Electrical Spinal Stimulation Promotes Long-term Recovery of Upper Extremity Function in Chronic Tetraplegia //IEEE Transactions on Neural Systems and Rehabilitation Engineering. -2018; Gad P. et al. Noninvasive activation of cervical spinal networks after severe paralysis //Journal of neurotrauma.—2018.-No. ja.).

The technical result using the invention is confirmed in the examples provided below. It should be understood that these and all the examples given in the application materials are not restrictive and are given only to illustrate this invention.

### Example 1

### Voluntary Arm Movements Increase the Amplitude of Involuntary Leg Movements Caused by Transcutaneous Electrical Stimulation of the Spinal Cord

The study was conducted to demonstrate the rhythmic arm movements facilitate the motor effects caused by TESSC.

### Methods

The study was carried out in Velikie Luki State Academy of Physical Education and Sports (VLSAPEC). Healthy volunteers—young men, employees and students of VLSAPEC (N=11, 20-35 years old)—took part in the study. Informed written consent of the subjects to participate in the trials was obtained in accordance with the principles of the Helsinki Declaration.

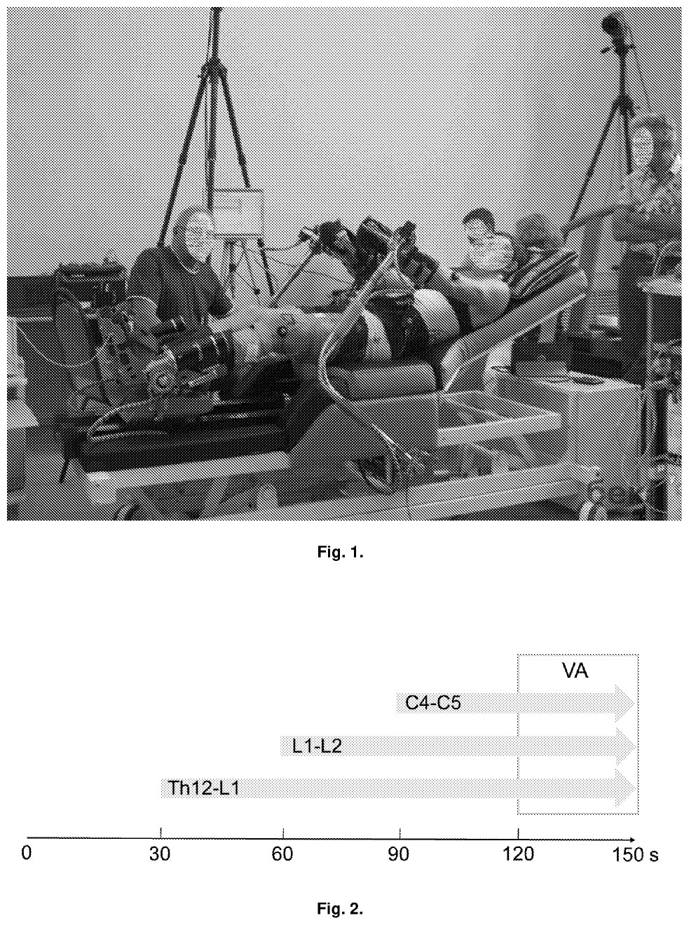

Referring to FIG. 1, the subjects were in semi-residential positions (FIG. 1) in a biomechanical training machine for neurorehabilitation of motor and visceral functions, Biokin-ES®. The training machine provides arbitrary or forced movements of the legs and/or arms. The leg movements are carried out in the hip, knee and ankle joints, imitating walking on the spot. The arm movements are carried out in the shoulder, elbow and wrist joints, leading to the shoulder and pushing away the levers in the sagittal plane. Transcutaneous stimulation of the spinal cord was performed in the subjects at the three levels: between the T12-L1, L1-L2, C4-05 vertebrae. The TESSC method has been described in detail earlier [Gerasimenko Y. et al. Initiation and modulation of locomotor circuitry output with multisite transcutaneous electrical stimulation of the spinal cord in non-injured humans //Journal of neurophysiology.—2014.-V. 113.-No 3. -P. 834-842.]. Five-channel programmable neurostimulator Biostim-5 (Grishin et al. 2017), which can be used for both diagnostic and therapeutic procedures using non-invasive electrical stimulation of the spinal cord, was used for TESSC. Rectangular monopolar pulses modulated with a carrier frequency of 5 kHz and a duration of 1 ms. were used. Pulse recurrence frequency is 30 Hz. The intensity of stimulation was selected individually, increasing the amplitude of the pulses from 5 mA gradually, achieving a motor response in all recorded leg muscles during stimulation at the lumbar level, focusing on the sensations of the subject during stimulation at the cervical level. The maximum current intensity was 70 mA during stimulation at the lumbar level and 30 mA at the cervical level. Electrical muscle activity (EMG) of the hip, m. biceps femoris, m. rectus femoris, and gastrocnemius muscles, m. gastrocnemius, m. tibialis anterior of both legs were recorded using skin electrodes of conductive plastic with an adhesive surface (Kendal). Elbow goniometers were used to record the arm movements. The hardware-software complex Mega (Finland) was used to record the EMG and limb movements. Video recording was also carried out using the Qualisys Medical video capture system, the reflector markers were attached to the body of the subjects from both sides at the bend points of the shoulder, hip, knee and ankle joints, as well as on the hallucis.

Referring to FIG. 2, Zero on the timeline corresponds to the beginning of the movement recording, T12-L1, L1-L2, C4-C5 correspond to moments of the beginning of the TESSC at each of the specified levels and VA is voluntary arm movements. The subject was in the training machine at rest state (FIG. 2), the TESSC was started after 30 sec at the level of the T12-L1 vertebrae, stimulation at the level of the L1-L2 vertebrae was added after another 30 seconds, at the level of the C4-C5 vertebrae—after another 30 seconds, stimulation was continued on three levels, the subject began to perform voluntary arm movements at the command of the experimenter after another 30 seconds.

The influence of the nature of movements and TESSC conditions on the kinematic parameters of leg movements and leg muscle activity was analyzed. The kinematic characteristics were calculated on the basis of the video recording, according to the coordinates of the markers located on the joints, the amplitude and speed of the step were calculated according to the coordinates of the movements of the hallux. Muscular activity was evaluated by the integral characteristic of EMG for each of the stimulation modes, for which purpose EMG recordings were filtered to get rid of the artifacts of the TESSC, inverted into a region of positive values and the area under the curve was determined in 30 sec. Changes in the integral characteristic in each of the modes were evaluated in relation to its value in the initial condition (i.e. for the first 30 sec of the study). The calculated relative values were averaged for all subjects, taking into account the results of the two legs. Mathematical data processing was performed using original programs and Microsoft Excel spreadsheets.

### Results

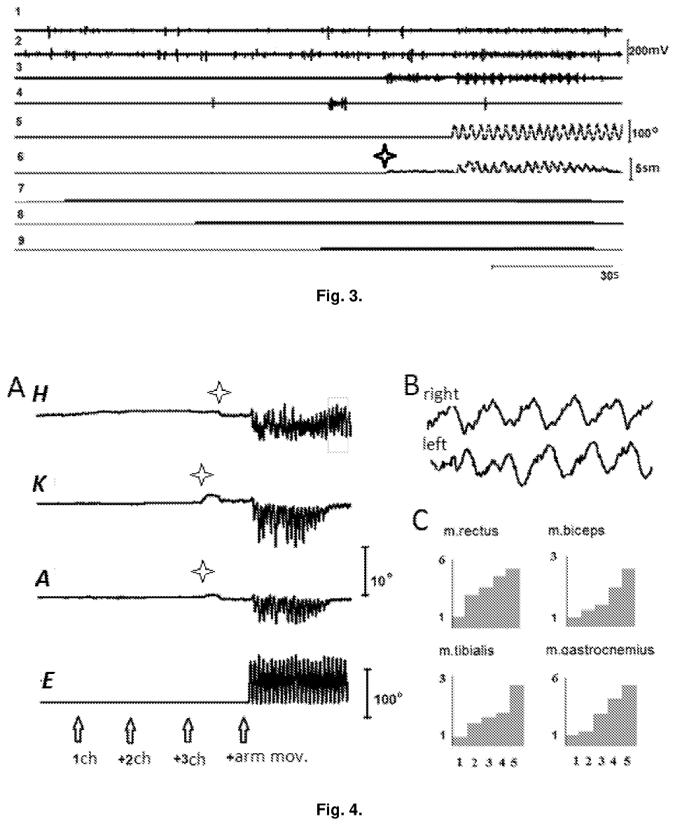

FIG. 3 illustrates recordings of a study of how arm movements affect the characteristics of involuntary leg movements caused by TESSC, obtained with the subject B.A. 1-4—EMG of the muscles of the right leg: anterior tibial muscle (1) (Latin m. tibialis anterior), gastrocnemius muscle (2) (Latin m. gastrocnemius), biceps of the thigh (3) (Latin m. biceps femoris) and rectus muscle of thigh (4) (lat. m. rectus femoris). 5—Angle changes in the right elbow joint. 6 - Changing the position of the right hallux. 7, 8 and 9—Stimulation channel activity marks at the level of the T12-L1, L1-L2 and the C5 vertebrate, respectively. An asterisk above line 6 indicates that the movement of the right leg has been initiated.

FIG. 4 illustrates recordings of an analysis of the effect of arm movements on the characteristics of movements caused by TESSC. A. —Angle changes in the right hip (H) knee (K), ankle (A), and elbow (E) joints at the beginning of TESSC at the level of the T12-L1 (1ch), L1-L2 (2ch), and C4-C5 (3ch) vertebrae, as well as at voluntary arm movements; asterisks indicate the initiation of movements in the joints of the subject B.A. B.—The reciprocity of movements in the hip joints of the right and left legs corresponds to the time interval allocated by the square in part A. C.—changes in the integral characteristics of the EMG activity of the leg muscle activity; a sequence of modes—on the abscissa axis: 1- resting state, 2, 3, 4—TESSC by the 1st (in the level of the T12-L1 vertebrae), 2nd (L1-L2) and 3rd (C4-C5) channels, respectively, 5 - performing voluntary movements of the arms. On the ordinate axis-integral characteristics of EMG for 30 s, referred to their value at the rest state, the average values for all subjects, relative values.

TESSC at the lumbar region of the spinal cord (electrodes placed on the T12-L1, L1-L2 vertebrae) did not cause leg movements (FIGS. 3, 4) in any of the subjects, but increased muscle activity (FIG. 4C). The absence of movement during increased muscle activity is associated with insufficient power of the muscle activity for shifting the heavy carriage of the training machine. Additional stimulation at the cervical region caused low-amplitude leg movements (FIG. 4A) in some subjects; the amplitude of movements of the markers on the hallucis was up to 4-5 cm when the carriages of the training machine were stationary (FIG. 3).

Voluntary rhythmic arm movements in the sagittal plane during the TESSC of the lumbar and cervical regions caused the rhythmic movements in the joints of the legs (FIG. 4A) in all subjects. Moreover, movements in the joints of the right and left legs were reciprocal (FIG. 4B), i.e. the induced movements were similar to the stepping movements. The activity of the muscles was maximal during voluntary arm movements (FIG. 4B).

### Conclusion

Voluntary arm movements in the rhythm of the step increase the total activity of the leg muscles and contribute to the initiation of rhythmic, step-like leg movements in the presence of multi-segmental TESSC.

### Example 2

### Spatio-Temporal Electrical Transcutaneous stimulation of the Spinal Cord Modulates the Characteristics of Human Stepping Movements

The study was conducted to demonstrate that it is possible to modulate the phase of swing in a stepping cycle (flexion), by stimulating the roots of the spinal cord at the level of the T11vertebrae periodically, in accordance to the step phase, and to modulate the stance phase (extensibility) by stimulating the roots of the spinal cord at the level of the L1 vertebrae periodically, in accordance to the step phase.

### Methods

The study was carried in the Velikie Luki State Academy of Physical Education and Sports (VLSAPEC). Healthy volunteers—young men, employees and students of VLSAPEC (N=9, 20-35 years old)—took part in the study. Informed written consent of the subjects to participate in the trials was obtained in accordance with the principles of the Helsinki Declaration.

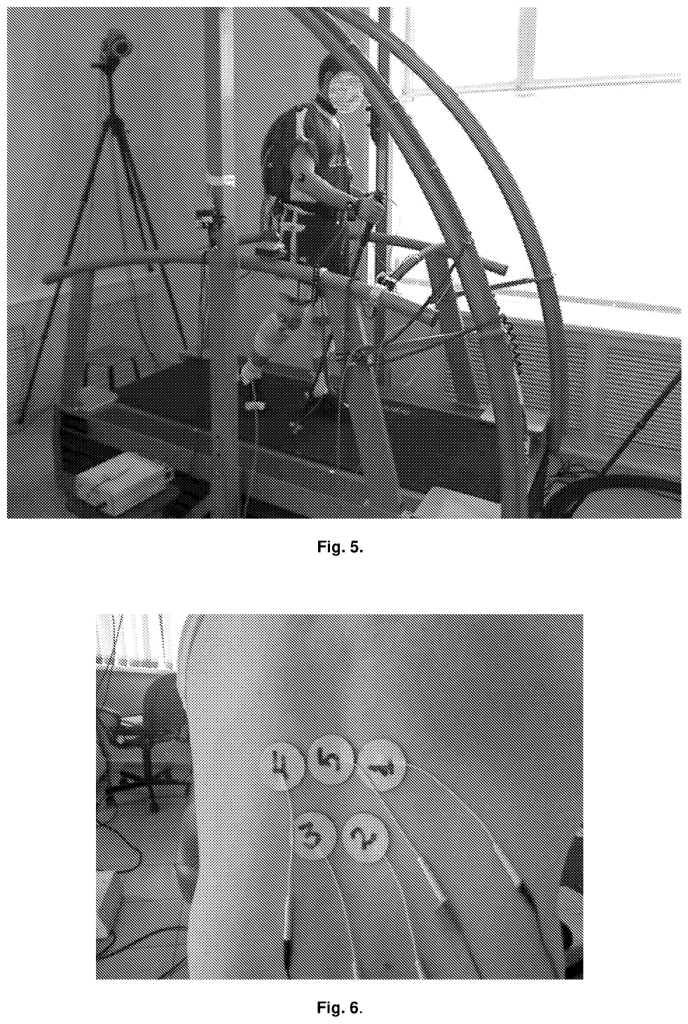

Referring to FIG. 5, the position of the subject during a study of how periodic TESSC applied to different roots of the spinal cord in different phases of the step (spatio-temporal TESSC) modulates the characteristics of human step movements is shown. The subjects were walking with the use of poles for Nordic walking on the treadmill of the training machine (FIG. 5). The speed of the treadmill was selected individually, the comfortable speed for all the test subjects was in the range of 1.8-2.0 km/h. Microswitches built in support ends of the poles for Nordic walking, are closed at leaning on a stick, the sync signal from closing is transferred to the switch which triggers channels of a programmed electrostimulator, for example Biostim-5™.

The TESSC method, the algorithm for selecting the intensity of stimulation and the characteristics of the pulses is the same as in Example 1. The differences are listed below.

FIG. 6 illustrates the location of cathodes in a projection of the spinal cord and the roots of the spinal cord during studies of the impact of spatio-temporal TESSC on the characteristics of human step movements. The electrode numbers correspond to the stimulation channels during the study. All the stimulating electrodes, five cathodes, were fixed in the lumbar spine area (FIG. 6). One cathode was located at midline of the spine, over the projection of the T11 vertebra, the remaining cathodes were fixed above the roots at the T11 vertebra, over the roots at the L1 vertebra on the left and on the right. Anodes—over the crest of the iliac bones, a pair of anodes common to all cathodes.

Stimulation by electrode 1 is periodic, activation of stimulation at the leaning on the contralateral (left) walking pole, deactivation at the detachment of the contralateral (left) walking pole.

Stimulation by electrode 2 is periodic, activation of stimulation at detachment of the contralateral (left) walking pole, deactivation at leaning on the contralateral (left) walking pole.

Stimulation by electrode 3 is periodic, activation of stimulation at detachment of the contralateral (right) walking pole, deactivation at leaning on the contralateral (right) walking pole.

Stimulation by electrode 4 is periodic, activation of stimulation at the leaning on the contralateral (right) walking pole, deactivation at the detachment of the contralateral (right) walking pole.

Stimulation by electrode 5 is continuous.

### The Procedure of the Stimulation

1. The subject walks on the treadmill for 15 sec;

2. Continuously stimulated at the level of the T11 vertebra (electrode 5 in FIG. 6) for 15 sec during stepping;

3. Periodic stimulation of the right root at L1 vertebra (electrode 1 in FIG. 6) for 15 sec during stepping and continuous stimulation at the level of the T11 vertebra;

4. Periodic stimulation of the right root at T11 vertebra (electrode 2 in FIG. 6) for 15 sec during stepping and continuous stimulation at the level of T11 vertebra and periodic stimulation of the right root at L1 vertebra;

5. Periodic stimulation of the left root at L1 vertebra (electrode 3 in FIG. 6) for 15 sec during stepping and continuous stimulation at the level of the T11 vertebra and periodic stimulation of the right roots at L1, T11 vertebrae;

6. Periodic stimulation of the left root at T11 vertebra (electrode 4 in FIG. 6) for 15 sec, during stepping and continuous stimulation at the level of the T11 vertebra and periodic stimulation of the right roots at L1, T11 vertebrae, and left root at L1 vertebra;

7. Stepping on a treadmill for 15 sec without stimulation.

Recording of electrical muscle activity and kinematics of movements using video analysis is the same as in Example 1.

### Results

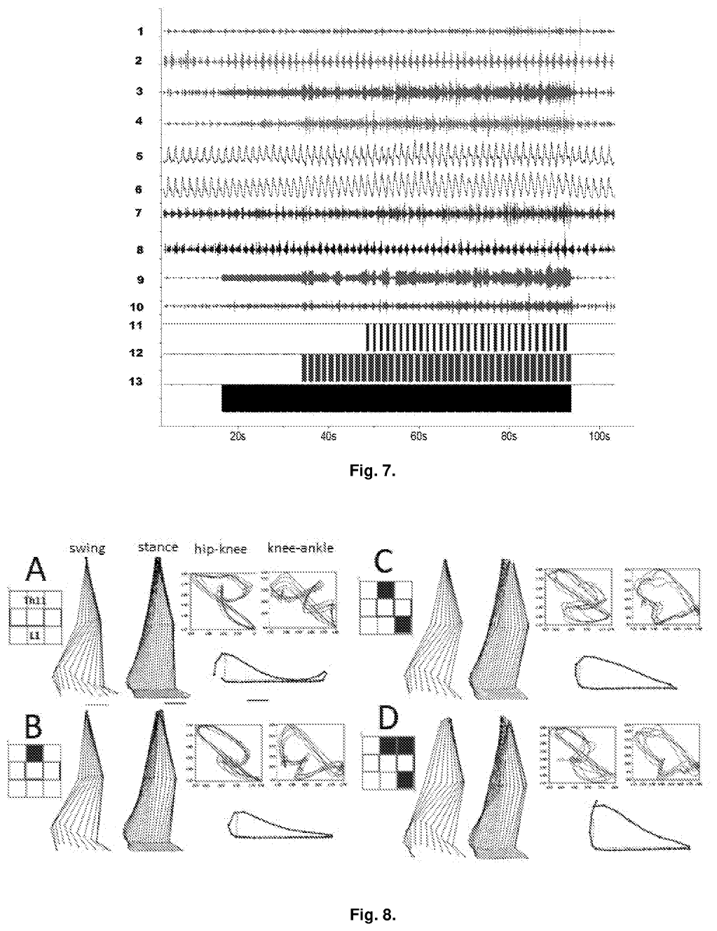

FIG. 7 presents a change in the EMG activity of the muscles of the legs and angles in the knee joint during the treadmill stepping of the subject D.G. at a speed of 2 km/h and periodic stimulation of the roots of the spinal cord according to the study protocol. Lines 1-4—EMG muscles on the right: tibialis anterior muscle (1) (Latin m. tibialis), gastrocnemius muscle (2) (Latin m. gastrocnemius), biceps of the thigh (3) (Latin m. biceps femoris) and rectus muscle of thigh (4) (Latin m. rectus femoris). Lines 5 and 6 are readings of the two-axis goniometer fixed on the right knee. Lines 7-10—EMG muscles on the left: tibialis anterior muscle (1) (Latin m. tibialis), gastrocnemius muscle (2) (Latin m. gastrocnemius), biceps of the thigh (3) (Latin m. biceps femoris) and rectus muscle of thigh (4) (Latin m. rectus femoris). Lines 11, 12 and 13—stimulation marks on electrodes 1, 2 and 5 in FIG. 6, respectively.

FIG. 7 shows an increase in the total muscle activity (lines 1-4 and 7-10) in response to each level of stimulation. An increase in the angle of the right knee (lines 5 and 6) is well noticeable during periodic stimulation on electrode 1 (line 11), which activates the extensors that provide the stance phase during stepping

FIG. 8 illustrates the trajectories of movements of the legs during treadmill stepping without stimulation and with continuous and periodic TESSC at the different levels. A—stepping on a treadmill without TESSC. B—stepping on a treadmill during continuous stimulation at the level of the T11 vertebra. C—stepping on the treadmill during continuous stimulation at the level of the T11 vertebra, periodic stimulation of the right root at L1 vertebra. D—stepping on a treadmill during continuous stimulation at the level of the T11 vertebra, periodic stimulation of the right roots at L1 and T11 vertebrae. Within each part of the figure, a stick diagram reconstructing the stance and swing phases is presented to the right of the active cathodes scheme; the coordination of angles in the femoral, knee and ankle joints is shown even more to the right, beneath them—the trajectory of the movement of the hallux in the sagittal plane; dashed right angle with the horizontal line equal to the length of the trajectory of the hallux in stance phase during the stepping without TESSC, and vertical line shows the maximum lift of the hallux during the stepping without TESSC.

The results of the analysis of the kinematics of locomotion are shown in FIG. 8. The effect of continuous TESSC at the level of the T11 vertebra (FIG. 8B), when a healthy person walks on a treadmill at a constant speed, is practically undetectable.

Changes in the kinematics of the stance phase at periodic (corresponding to the stance phase) stimulation of the right root of the spinal cord at the level of L1 are not evident at first sight and this is due to the fact that the treadmill sets the rhythm of stepping. But the effect can be seen even in such conditions.

Extreme values and range of angle changes in right hip, knee and ankle joints under conditions A-D in FIG. 8 are shown in Table 1 (in angular degrees).

TABLE 1

| | A | | | B | | | C | | | D | | |
|---|---|---|---|---|---|---|---|---|---|---|---|---|
| | min | max | max − min | min | max | max − min | min | max | max − min | min | max | max − min |
| hip | 154 | 180 | 26 | 157 | 178 | 21 | 157 | 178 | 21 | 156 | 180 | 24 |
| knee | 127 | 180 | 53 | 129 | 179 | 50 | 122 | 180 | 58 | 111 | 180 | 69 |
| ankle | 94 | 118 | 24 | 94 | 120 | 26 | 91 | 121 | 30 | 90 | 124 | 34 |

The range of changes in the angle of the knee (FIG. 8, A-C) at normal stepping was 127-180 angular degrees, at continuous TESSC stimulation at the level of the T11-129-179 angular degrees, at the stimulation of the root at L1 vertebra—122-180 angular degrees, that is, an increase in the range of angle changes in the knee joint at the activation of extensors in the stance phase, the increase in the range is due to greater flexion in the knee joint. The range of ankle angle changes has also increased (FIG. 8 A-C), at normal stepping it was 94-118 angular degrees, at continuous TESSC at the level of the T11 -94-120 angular degrees, at the stimulation of the root at L1 vertebra—91-121 angular degrees, i.e. an increase in the range of angle changes in the knee joint at the activation of extensors in the stance phase was recorded, the increase in the range occurs at the expense of an increase in the back flexion of the foot. The stepping during the stimulation of the right root at L1 vertebra presented as if the subjects were squatting on the stimulated right side.

The elevation of the foot above the surface increased markedly with periodic (corresponding to the swing phase) stimulation of the right root of the spinal cord at the level of the T11 vertebra: the contour describing the trajectory of the hallux of the right foot elevated significantly higher than with all other stimulation options (see the dashed triangle corresponding to the parameters of the stance and swing phases during locomotion without stimulation). This demonstrates the work of the flexors whose activation is required during the swing phase. The range of changes in the knee joint increased even more than with the stimulation of the root at L1vertebra (111-180 angular degrees) and also the range in the ankle joint (90-124 angular degrees) increased (FIG. 8). The flexion in the knee and the extension in the ankle joint increased, externally, this stepping presented as an accentuated elevation of the right knee during walking.

A significant asymmetry of the dynamic characteristics of stepping performance became apparent during the stimulation of the right roots. Changes in the maximum instantaneous step speed when stepping on a treadmill at a speed of 2 km/h for the right and left legs are shown in Table 2.

Measurements by marker on the hallux averaged over all locomotion cycles for 15 sec. The maximum instantaneous step speed on the right side was different from this indicator on the left by 17-33% at asymmetric stimulation and by 8-9% at symmetric TESSC or in the absence of stimulation.

| | | | | Locomotion + | Locomotion + |
|---|---|---|---|---|---|
| | | | Locomotion + | TESSC T11 + | TESSC T11 + |
| | | | TESSC T11 + | pTESSC L1, | pTESSC L1, T11, |
| | Normal | Locomotion + | pTESSC L1, | right + pTESSC | right + pTESSC |
| | locomotion | TESSC T11 | right | T11, right | L1, T11, left |
| Speed on the right (m/s) | 2.4 | 2.2 | 2.1 | 2.4 | 2.6 |
| Speed on the left (M/Ceκ) | 2.6 | 2.4 | 2.8 | 2.8 | 2.8 |
| Difference of the speeds (%) | 8.3 | 9.1 | 33.3 | 16.6 | 7.7 |

### Conclusion

It is shown that TESSC applied over the roots of the spinal cord that activate extensor motor pools in the stance phase of the step, and the flexor motor pools in swing phase of the step, respectively, allowed modulation of the kinematic and dynamic characteristics of the step, and to control human gait.

Although the invention has been described with reference to the disclosed embodiments, it should be obvious to specialists in the art that the specific detailed studies described are only for the purpose of illustrating this invention and should not be construed as limiting the scope of the invention in any way. It should be clear that it is possible to make various modifications without departing from the essence of this invention.

1. A method of regulation and restoration of independent stepping for patients with traumatic injuries and/or diseases of the spinal cord and/or brain using transcutaneous electrical stimulation of the spinal cord, including simultaneous continuous stimulation of the spinal cord at least at the level of the T11-T12 vertebrae and spatio-temporal and spatio-selective stimulation of the roots of the spinal cord at the level of the T11 and L1 vertebrae.

2. The method in accordance to claim 1, in which the triggering and cessation of transcutaneous electrical stimulation of the spinal cord is performed through external control.

3. The method in accordance to claim 2, in which one of the movements performed to control with the device for triggering or cessation of stimulation can be chosen as an external control: movement of at least one intact upper or lower limb, head movement, shoulder lift, torso movement.

4. The method in accordance to claim 1, in which the trigger of spatio-temporal and spatio-selective stimulation of the roots of the spinal cord at the level of the T11 and L1vertebrae is the motion of the intact upper or lower limb.

5. The method in accordance to claim 1, in which spatio-temporal and spatio-selective stimulation of the roots of the spinal cord depends on the phase and speed of movement of the upper limb and/or lower limb.

6. The method in accordance to claim 1, in which current intensity in transcutaneous electrical stimulation is selected individually depending on the excitability and threshold of the motor response and pain sensitivity of the patient.

7. The method in accordance to claim 6, in which the monopolar rectangular pulses or bipolar rectangular pulses with a modulation frequency in the range from 5 kHz to 10 kHz are used for transcutaneous electrical stimulation of the spinal cord, with a stimulation frequency for continuous stimulation of the spinal cord at least at the level of the T11-T12 vertebrae chosen in the range from 30 Hz to 45 Hz, the stimulation frequency for stimulation of the roots of the spinal cord at the level of the L1 vertebrae is chosen in the range from 10 Hz to 30 Hz, the stimulation frequency for stimulation of the roots of the spinal cord at the level of the T11 vertebrae is chosen in the range from 30 Hz to 50 Hz.

8. The method in accordance to claim 1, in which the stimulations are performed in patients with hemiparesis.

9. The method in accordance to claim 8, in which the patient walks over ground or moving treadmill with the upper limb leaning on a fixed support or with partial compensation of body weight by means of a suspension system during continuous stimulation of the spinal cord and spatio-temporal and spatio-selective stimulation of the roots of the spinal cord.

10. The method in accordance to claim 9, including the following successive steps:
1) triggering and activation of continuous stimulation at least at the level of the T11-T12 vertebrae and triggering of spatio-temporal and spatio-selective stimulation of the roots of the spinal cord at the level of the T11 and L1 vertebrae by means of external control, which initiates the beginning of stimulation,
2) activation of stimulation of the roots of the spinal cord on the lesion side at the level of the L1 vertebrae at the moment when the intact lower limb is detached from the surface and swung forward,
3) deactivation of the stimulation of the roots of the spinal cord on the lesion side at the level of the L1 vertebrae with simultaneous activation of the stimulation of the roots of the spinal cord on the lesion side at the level of the Tn vertebrae at the moment of placing the intact lower limb on the surface,
4) stimulation of the roots of the spinal cord on the lesion side at the level of the Tn vertebrae when the paretic lower limb is detached from the surface and swung forward,
5) deactivation of the stimulation of the roots of the spinal cord on the lesion side at the level of the Tn vertebrae at the moment of placing the paretic lower limb on the surface,
6) repeating steps 2)-5) any amount of times,
7) cessation and deactivation of continuous stimulation of the spinal cord at least at the level of the T11-T12 vertebrae and cessation of spatio-temporal and spatio-selective stimulation of the roots of the spinal cord at the level of the T11 and L1 vertebrae due to external control, which stops stimulation.

11. The method in accordance to claim 10, in which continuous stimulation of the spinal cord maybe additionally performed at the level of C5-C6 vertebrae.

12. The method in accordance to claim 8, in which the patient performs over ground stepping or stepping on a moving treadmill during continuous stimulation of the spinal cord and spatio-temporal and spatio-selective stimulations of the roots of the spinal cord using a walking aid on which the patient's intact upper limb leans.

13. The method in accordance to claim 12, in which the walking aid is selected from: a walking stick, a cane, a crutch.

14. The method in accordance to claim 12, in which the patient performs stepping movements using a suspension system.

15. The method in accordance to claim 12, including the following successive steps:
1) triggering and activation of continuous stimulation at least at the level of the T11-T12 vertebrae and start of spatio-temporal and spatio-selective stimulation of the roots of the spinal cord at the level of the T11 and L1 vertebrae by means of external control, which initiates the beginning of stimulation,
2) activation of stimulation of the roots of the spinal cord on the lesion side at the level of the L1 vertebrae at the moment the intact lower limb is detached from the surface and swung forward,
3) deactivation of the stimulation of the roots of the spinal cord on the lesion side at the level of the L1 vertebrae at the moment of placing the intact lower limb on the surface,
4) activation of stimulation of the roots of the spinal cord on the lesion side at the level of the T11 vertebrae at the moment of lifting of the walking aid from the surface and transferring its forward due to the motion of the intact upper limb; placing the walking aid on the surface;
5) stimulation of the roots of the spinal cord on the lesion side at the level of the T11 vertebrae when the paretic lower limb is detached from the surface and swung forward,
6) deactivation of stimulation of the roots of the spinal cord on the lesion side at the level of the T11 vertebrae at the moment of placing the paretic lower limb on the surface,
7) repeating steps 2)-6) any amount of times,
8) cessation and deactivation of continuous stimulation of the spinal cord at the level of the T11-T12 vertebrae and at the level of the C5-C6 vertebrae and cessation of spatio-temporal and spatio-selective stimulation of the roots of the spinal cord at the level of the T11 and L1vertebrae occurs due to external control, which stops the stimulation.

16. The method in accordance to claim 15, in which continuous stimulation of the spinal cord can be performed additionally at the level of the C5-C6 vertebrae.

17. The method in accordance to claim 1, in which transcutaneous electrical stimulation is performed using a multi-channel stimulator.

18. The method in accordance to claim 17, in which at least one recording device is used to detect:
the contact of the lower limb or the walking aid, on which the intact upper limb leans, with the surface,
the detachment of the lower limb or the walking aid, on which the intact upper limb leans, from the surface;
and at least one recording device transmits the control signal to the stimulator when the above events are detected.

19. The method in accordance to claim 18, in which the stimulator contains
at least one storage device
and one or more programs that are loaded into at least one of the above-mentioned storage devices, with one or more programs containing instructions for:
triggering and cessation of stimulation in dependence of the control signal received from the device for triggering or cessation of stimulation during external control;
activation and deactivation of continuous stimulation by regulating the supply of electrical current to the corresponding electrodes, in dependence of the control signal received from the device for triggering or cessation of stimulation during external control;
activation and deactivation of spatio-temporal and spatio-selective stimulations of the roots of the spinal cord by regulating the supply of electrical current to the corresponding electrodes, in dependence of the control signal received from at least one device for recording the detection of contact and/or detachment of the intact and/or paretic lower limb and/or walking aid on which the intact upper limb leans.

20. A spinal neuroprosthesis for the regulation and restoration of independent stepping in patients with traumatic injuries and/or diseases of the spinal cord or brain, including
a multi-channel stimulator for transcutaneous electrical stimulation of the spinal cord that includes at least one storage device and one or more programs that are loaded into at least one storage device, with one or more programs containing instructions for the implementation of the method according to claim 1
electrodes or electrode array connected to the stimulator,
at least one recording device to detect the contact and/or detachment of the intact and/or paretic lower limb and/or the walking aid on which the intact upper limb leans during stepping,
a device for triggering or cessation of stimulation.

21. The spinal neuroprosthesis in accordance to claim 20, in which at least one recording device may be selected from: surface contact sensor, surface contact sensor of the walking aid.

22. The spinal neuroprosthesis in accordance to claim 20, in which the device for triggering or cessation of stimulation may be made in the form of an electromechanical switch or radio-frequency switch.

23. The spinal neuroprosthesis in accordance to claim 20 may includes an acceleration sensor and/or angular velocity sensor and/or a joint angle sensor.
